(19) United States
(12) Patent Application Publication
(10) Pub. No.: US 2009/0247991 A1
(43) Pub. Date: Oct. 1, 2009

(54) Operating room instrument

(75) Inventors: Ancil W. Sigman, Reno, NV (US); Stuart D. Alderman, Reno, NV (US)

Correspondence Address: SILVERSKY GROUP LLC, 5422 LONGLEY LANE, SUITE B, RENO, NV, 89511, US

(73) Assignee: Squint-X, LLC, Reno, NV (US)

(21) Appl. No.: 12/079,561

(22) Filed: Mar. 27, 2008

(51) Int. Cl. A61B 17/00

(52) U.S. Cl. 606/1

(57) ABSTRACT

The present invention is directed to an instrument for use in operating rooms/theatres during surgical procedures, which enables a sterile technician to assemble, mount and utilize a sterilized instrument, such as a magnifier, without the assistance of a non-sterile technician. The instrument is comprised of one or more clamps pre-mounted to a lower portion of operating tables within the operating theatre and a series of rods that can be quickly assembled and inserted into the clamps to create a stand and, separately, an arm for receiving a variety of different tools useful during surgery.

### BRIEF DESCRIPTION OF THE INVENTION

The present invention is directed to an instrument for use in operating rooms/theatres during surgical procedures, which enables a sterile technician to assemble, mount and utilize a sterilized instrument, such as a magnifier, without the assistance of a non-sterile technician. The instrument is comprised of one or more clamps pre-mounted to a lower portion of various tables within the operating theatre and a series of rods that can be quickly assembled and inserted into the clamps to create a stand and, separately, an arm for receiving a variety of different tools useful during surgery.

### CROSS-REFERENCES TO RELATED APPLICATIONS

Not Applicable.

### STATEMENT AS TO THE RIGHTS TO INVENTIONS MADE UNDER FEDERALLY SPONSORED RESEARCH OR DEVELOPMENT

Not Applicable.

### REFERENCE TO A “SEQUENCE LISTING,” A TABLE, OR A COMPUTER PROGRAM LISTING APPENDIX SUBMITTED ON A COMPACT DISK

Not Applicable.

### BACKGROUND OF THE INVENTION

A variety of personnel typically work within an operating room or theatre during a surgery or medical procedure to provide medical services to a patient. The surgeons, nurses, anesthesiologists and, typically, some technicians are “sterile,” meaning that they have been made as free as possible of infectious microorganisms and other contaminants prior to entering the operating room and attempt to maintain that sterile condition during their work on the patient. This does not mean, however, that the entire operating room is sterile or every team member in the room is sterile or completely sterile. Rather, a “sterile field” is established within the room that covers a three dimensional area within the room that extends from the chest of the sterile workers (and about two inches above the elbow of each arm) and at and above some level of the patient. Only the top surfaces of tables are considered sterile. Anything extending over the edge or hanging over the edge of the table is considered non-sterile. Non-sterile team members (also known as a “circulator”) within the operating room typically perform any work outside of the sterile field, such as operating equipment, picking up anything dropped on the floor, documenting equipment and device usage, etc.

Over the years, a body of knowledge and set of procedures have been developed to ensure, to the maximum extent feasible, that foreign substances including pathogens, proteinaceous materials, certain chemicals, particulates, and other specific materials are not introduced into the sterile field and therefore possibly into the patient. At the same time, an increasing number of team members operating within the sterile field are called upon to identify, handle, assemble and otherwise process a variety of parts and devices used in conjunction with the surgery/procedure or to be implanted within the patient.

These devices are often quite small and their identification, selection, use and assembly is exacting. In order for surgical team members to keep careful track of every part/device used during surgery, and to enable patients with subsequently discovered defective implanted parts to be identified, all of these parts/devices must be identified and cataloged during surgery. This is typically done by having one team member responsible for reading small serial numbers printed or etched on the parts/devices, while another team member records the recited numbers. Present solutions for providing sterile team members with the ability to handle and identify these devices are: (1) either inconvenient and difficult to use (such as unaided identification or even hand held magnifying instruments); (2) expensive, awkward and overly technical (such as head mounted optical magnifiers); or (3) require the assistance of a non-sterile circulator to mount or move the instruments for the sterile team members. The present solutions are inadequate, increase costs, increase the risk of error, generate significant team member stress, and reduce the quality of surgical procedures.

### BRIEF DESCRIPTION OF THE SEVERAL VIEWS OF THE DRAWING

FIG. 1 is a partially broken, perspective view of the present invention installed on an operating room table;

FIG. 2 is several perspective views of the individual components of the present invention as illustrated in FIG. 1;

FIG. 3 shows an alternative embodiment of the tool arm illustrated in FIGS. 1 and 2; and

FIG. 4 is several plan views and an elevation view of the clamp halves illustrated in FIG. 2.

### DETAILED DESCRIPTION OF THE INVENTION

The present invention is directed to an instrument for use in operating rooms during surgical procedures, which can be assembled, mounted and utilized by a sterile team member without requiring the sterile team member to leave the sterile field and without requiring the assistance of a non-sterile team member. The instrument is comprised of one or more clamps pre-mounted to a lower portion of a table within the operating room and a series of rods that can be quickly assembled by a sterile team member and inserted into the clamps without breaching the sterile field so as to create a stand and, separately, an arm for receiving a variety of different tools useful during surgery.

The instrument of the present invention, especially when equipped with a magnifier tool, has a number of benefits that will become apparent during the course of this detailed description. Because the parts of the instrument can be rapidly assembled by a sterile team member, the instrument of the present invention speeds up operating room procedures and lowers the cost of surgery. When equipped with the magnifier tool, the instrument and tool of the present invention reduces errors in the identification and documentation of parts/devices, reduces errors in the operation and manipulation of such parts/devices, reduces emotional and ocular stress for team members responsible for part/device identification, reduces liability due to potential errors, and increases the success and quality of outcomes for patients.

As will be further described below with reference to the figures, the sterilize-able parts of the instrument and tool are easily disassembled, cleaned and sterilized. Once in the operating room, the instrument and tool can be rapidly assembled and installed by a single sterile team member without breaching the sterile field. Furthermore, the design and materials from which the instrument and tool are constructed are durable and eliminate the source of most malfunctions that would be likely to occur with such instruments and tools. The simple, but effective, magnifier tool is easy to use and significantly reduces the possibility for errors during surgical procedures.

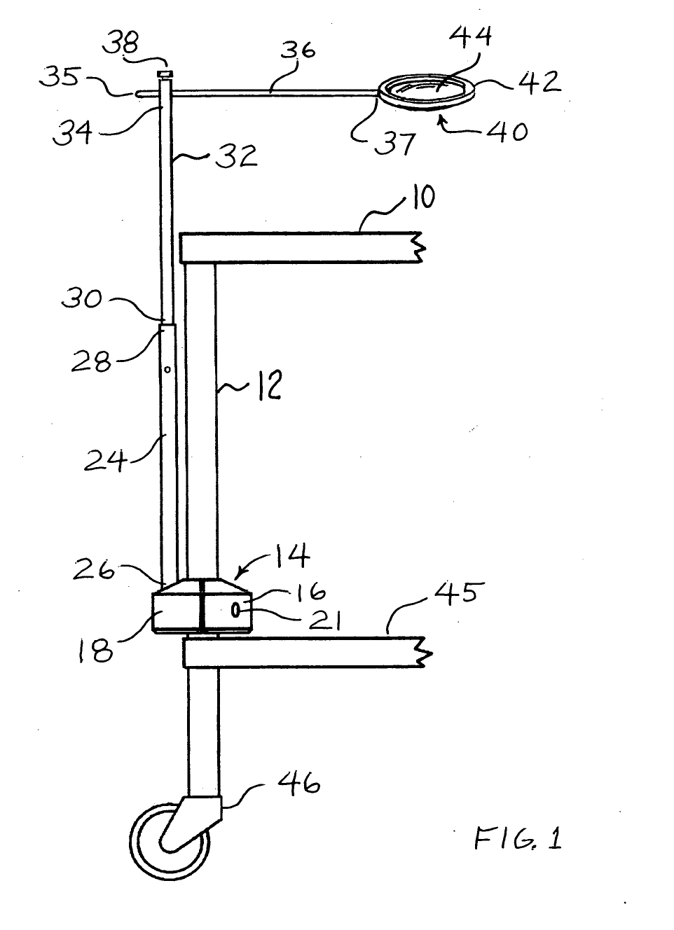

With reference now to FIG. 1, a preferred embodiment of the present invention is illustrated as installed on an operating room table 10 of some type. Operating room tables come in a number of different forms. Some tables are used to hold patients during surgery while other tables are used to hold instruments, devices, parts, etc. One type of this latter table is referred to as a “Mayo stand,” which typically has one or two legs and is capable of having its upper surface positioned close to or over a patient during surgery. The missing leg(s) makes it possible for the table to be moved over the patient. Another type of table typically used to hold instruments, devices, parts, etc., as well as being used for assembly and adjustment of various parts and devices, is called an instrument table or a “back table.” Instrument tables usually have four legs, one in each corner and are usually mounted on wheels that allow the tables to be easily moved. Other types of tables, with different numbers of legs and supports, such as cross bars and lower rails, could also be utilized. Table 10 in FIG. 1 (shown as partially broken) is a four-legged table, but regardless of the number of legs involved, the present invention could be utilized on any type of operating room table.

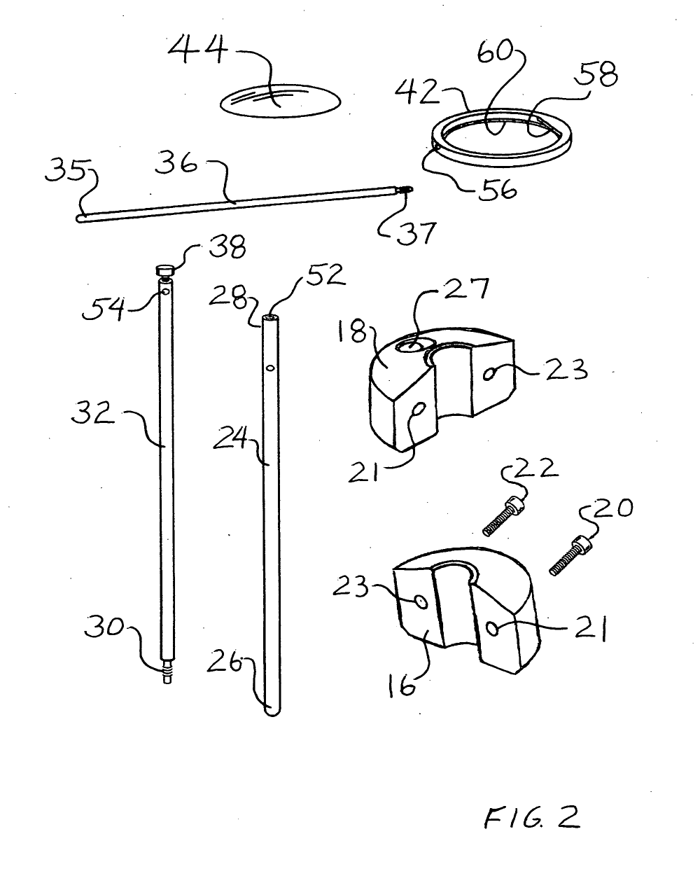

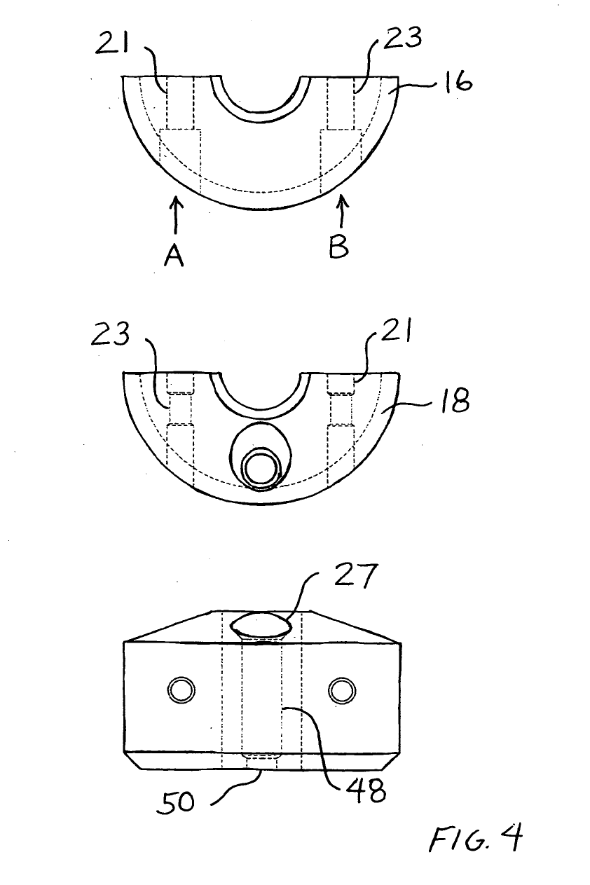

Attached to the leg 12 of table 10 is a presently preferred embodiment of table clamp 14, which is comprised of two halves 16 and 18, and which are held together by bolts 20 and 22 (both shown in FIG. 2), inserted through bolt slots 21 and 23 (slot 23 is illustrated in FIG. 2). Rather than being attached to one or more legs, the clamp could also be attached to a cross bar, lower rail, or other form of support that is within the non-sterile field under the top of the table. A lower rod 24 has one end 26 inserted into an opening 27 (illustrated in FIGS. 2 and 4) in clamp half 18 and its other end 28 connected to a first end 30 of upper rod 32. The second end 34 of upper rod 32 has an opening 54 (illustrated in FIG. 2) therein for receiving a smooth end 35 of a tool arm 36. The second end 34 also includes a thumb screw 38 for securing the tool arm against the upper rod 32. Tool arm 36 also includes a threaded end 37 (further illustrated in FIG. 2) that is screwed into a bracket for holding a tool, such as magnifier tool 40, which is comprised of lens ring 42 and lens 44.

The instrument of the present invention is comprised of a number of parts so it can be disassembled, cleaned and sterilized in an autoclave, a device which utilizes superheated steam or chemicals to sterilize objects, such as surgical equipment and waste products. Once the parts of the instrument (and any tool to be used) have been sterilized, they are removed from the autoclave and carried into the operating room, where they can be reassembled when needed. Disassembly is also important because the instrument needs to be large enough that one end of the instrument can be mounted in the table clamp 14 while the other end of the instrument extends high enough above the surface of the table 10 to be comfortably used by a sterile team member. Given this requirement, if the instrument was comprised of only a single rod instead of lower rod 24 and upper rod 32, the instrument might not fit within the typical autoclave. In a presently preferred embodiment, the stand is comprised of lower rod 24, which is 17 inches long with a ½ inch diameter, and upper rod 32, which is also 17 inches long from first end 30 to the top of thumb screw 38, with a 7/16 inch diameter. When the upper rod 32 is inserted into the lower rod 24, the useable length of the stand is about ¾ inch shorter.

The combined useable length (about 33¼ inches) of the two rods 24 and 32 is also important for another reason, the ease with which one end 26 can be inserted into clamp half 18. Keeping in mind that the instrument must be capable of singly being used by a sterile team member without any assistance from a circulator (either inside or outside of the sterile environment of the operating room), it is important that the combined length of the two rods 24 and 32 be such that the sterile team member can easily position the one end 26 into the clamp half 18 by only holding the upper end of upper rod 32 and without moving their hands or any other sterile portion of their body out of the sterile field, i.e., below the upper surface or portion of the table 10, and into the non-sterile filed, the lower portion of the table.

Although a presently preferred embodiment is recited with respect to the length and diameter of the rods, the present invention is not restricted to that embodiment. Shorter or longer rods, with smaller or larger diameters, or a different number of rods could readily be utilized and keep within the spirit of the invention described herein. For example, operating room tables could be lower or higher than the operating room or Mayo-type stands described herein, requiring possibly different rod lengths. At the same time the clamp 14 could also be mounted higher or lower on a table leg 12 to accommodate different rod lengths.

As illustrated in FIG. 1, the clamp 14 is illustrated as being mounted on the leg 12 toward a lower shelf 45. Obviously, the clamp 14 could be mounted even lower on the leg 12, such as closer to the wheel assembly 46, or higher on the leg 12, such as closer to the top of the table. In other words, the clamp 14 could be mounted almost anywhere as long as the clamp 14 remains below the sterile field and opening 27 can be readily accessed by the sterile team member when inserting the lower rod 24 into the opening 27 from within the sterile field. For example, it might be desirable to have a clamp 14 mounted on every leg of the tables in the operating room or on two cattycorner legs of a four leg table, one clamp at one height and the other clamp at a second height so the height of the rods could be adjusted by simply rotating the table 10 and without requiring a non-sterile team member to adjust the clamp or requiring any sterile team member to reach out of the sterile field.

The clamp 14 is better illustrated with reference now to FIGS. 2 and 4. The bolts 20 and 22 would be inserted into bolt slots 21 and 23, respectively, of clamp half 16 in the direction of the arrows A and B. The bolt slots 21 and 23 are smooth bored so the shafts of the bolts 20 and 22 can pass through unobstructed, but the bolt heads would be blocked when they reached the narrower portion of the bolt slots 21 and 23. The threaded shafts of the fully inserted bolts 20 and 22 would then enter bolt slots 21 and 23 of clamp half 18 and mate with the threaded interior of the bolt slots in clamp half 18. Alternatively, a nut could be inserted into each of the bolt slots of clamp half 18 and be used to engage and secure the bolts. Although the clamp 14 represents a presently preferred embodiment, the exact structure of the clamp 14 is not critical and could be replaced with many other types of clamps, as long as the clamp 14 could attach to the leg 12 of the table 10, included an opening or self operating brace of some type (such as a clip, gripper or stud arrangement, etc.) for holding the lower rod 24, and was readily accessibly by a sterile team member while that team member remained within the sterile field.

The preferred dimensions of the opening 27 are also further illustrated in FIG. 4, which shows that opening 27 has a funnel shaped mouth that is designed to guide the one end 26 of lower rod 24 into the opening, as well as a channeled portion 48 that causes the one end 26 to be directed toward and securely seated on the bottom 50 of the opening 27. The one end 26 of lower rod 24 is rounded which further assists in guiding the lower rod 24 into the channeled portion 48. This arrangement improves the stability of the lower rod 24 when in use, while still enabling the lower rod to be easily removed if necessary, but other types of holes and braces are possible.

At the other end 28 of lower rod 24, a threaded hole 52 is formed for receiving the threaded first end 30 of upper rod 32. Upper rod 32 includes a smooth through hole 54, through which smooth end 35 of tool arm 36 is inserted. Once the tool arm 36 is moved to its desired position relative to the upper rod 32, thumb screw 38 would be tightened to hold the tool arm 36 in position. The threaded end 37 of tool arm 36 would be attached to a tool, in this case magnifier tool 40. As illustrated in FIG. 2, the threaded end is screwed into the ring hole 56 of the lens ring 42, but the magnifier tool 40 and all types of other tools could be attached, affixed or otherwise secured to the tool arm 36 in an almost unlimited number of ways.

Other tools and attachments could be used with this device which could include, but are not limited to, positioning tools, assembly jigs, metering and measuring devices, alternate optical devices, cutting and fabricating tools, etc.

With respect to the present invention, in addition to securing the lens ring 42 to the tool arm 36, the threaded end 37 serves the additional function of helping to retain the lens 44 in place. On the opposite side of the interior of the lens ring 42 from the ring hole 56 is a protruding area 58 under which an edge of the lens 44 would be inserted and retained against the lip 60 of the lens ring 42. When the threaded end 37 is fully inserted into the ring hole 56, the end of the threaded end sticks out into the interior of the lens ring 42 just far enough to form an opposing retainer to the protruding area 58. When the lens 44 is held in place by the lip 60, the protruding area 58 and the threaded end 37, it is fairly loosely held in place. The lens 44, which is preferably plastic, is loosely held within the lens ring, which is preferably metal, so there is sufficient room for the lens 44 to thermally expand when the magnifier tool is subjected to the heat generated by an autoclave. This enables the magnifier tool to be assembled prior to being autoclaved, versus being autoclaved in pieces and then assembled after being sterilized.

The ability to easily disassemble, sterilize and reassemble the present invention is a feature. Prior to being introduced to a sterile environment, as noted above, the upper rod 32, lower rod 24, tool arm 36 and tool 40 would be disassembled from one another and placed in an autoclave for sterilization.

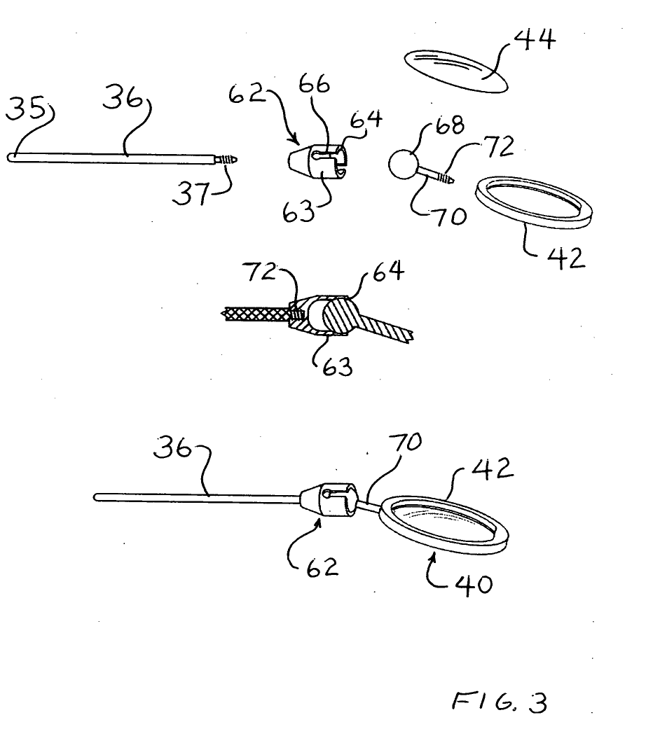

An alternative embodiment of the tool holding portion of the present invention is shown in FIG. 3. In this embodiment, tool arm 36 is attached to a swivel device 62 rather than directly to the tool 40. The swivel device 62 is a ball and socket device that enhances a user's ability to position the tool 40 at various angles. The swivel device 62 has a socket body 63 with a lip 64 and one or more slots 66. The entire swivel device 62 could be comprised of a flexible material which allows the ball 68 of the swivel arm 70 to be inserted into the socket body 63. Alternatively, just the socket body 63 of the swivel device 62 could be formed of a flexible material, while the remainder of the swivel device 62 is formed of a more rigid material.

The flexibility of the socket body 63 is necessary because the area within the socket body 63 is smaller than the area of the ball 68, thereby requiring the ball 68 to be forced into the socket body 63 and the socket body 63 to flex in order to receive the ball 68. The resulting pressure asserted by the socket body 63 against the ball 68 causes the ball 68 to be held firmly in any position it is placed. The slots 66 can be configured to help provide flexibility to the swivel device 62. The threaded end 72 of swivel arm 70 would then be secured to the tool 40.

The alternative embodiment shown in FIG. 3 provides additional advantages in that it can be quickly disassembled, sterilized and reassembled. It provides a minimum number of additional parts and the design is simple, allowing the parts to be easily cleaned and sterilized. As a further alternative, the socket body 63 of the swivel device 62 could be a simple seat, without a lip extending over the ball 68 and without the slots 66, within which the ball 68 is placed. A separate cylindrical cap (not shown), with a large central opening on one end and a small central opening on the other end (formed by an end wall of the cap), could then be placed over the swivel arm 70 and the ball 68 to hold the ball 68 in place. The threaded end 72 of the swivel arm 70 would enter the large central opening and exit the small central opening, while the ball 68 would enter the large central opening and be stopped by the end wall at the other end of the cap. Thus, the ball 68 would be held in place against the seat by the cap, and the cap would be held in place against the ball 68 when the threaded end 72 was screwed into the tool 40. Naturally, many other alternative embodiments of the tool holding portion of the invention are possible and the spirit and scope of the present invention is not limited to just those embodiments disclosed herein.

The advantages of the present invention, as partially noted above, are many. The present invention reduces the emotional stress of sterile team members during surgery as they are no longer required to squint or seek alternative methods in order to read tiny identifications on parts and objects and assemble tiny and often intricate devices. Sterile team members are no longer required to either work with a non-sterile team member to set up an instrument or assemble and operate a more complicated instrument by themselves. This also speeds up performance and increases accuracy within the operating room/theatre. Hospitals and surgical centers benefit by reducing time spent performing a number of intricate procedures; they increase the quality of care they are able to provide; they improve working conditions for their employees; and they decrease liability by reducing a potential source of errors.

While the present invention has been illustrated and described herein in terms of a preferred embodiment and several alternatives associated with sterile operating room instruments, it is to be understood that the various components of the combination illustrated, and the combination itself, can have a multitude of additional uses and applications. Accordingly, the invention should not be limited to just the particular description and various drawing figures contained in this detailed description that merely illustrate a preferred embodiment and application of the principles of the invention.

What is claimed is:

1. An instrument for use inside a sterilized environment, including a sterile field and a non-sterile field, by a person including a sterile portion, comprising:
a first component operative to be affixed to a table located inside the sterilized environment, the table having an upper portion inside the sterile field and a lower portion inside the non-sterile field, the first component being affixed to the lower portion; and
a second component including a shaft, an arm and a tool, the tool being operative to be affixed to the arm, the arm being operative to be affixed to the shaft, the shaft being operative to be held by the sterile portion of the person and mounted to the non-sterile component without requiring the sterile portion to enter the non-sterile field, and without requiring assistance from a non-sterile person either located inside the sterilized environment or located outside of the sterilized environment.
